(19) United States
(12) Patent Application Publication
(10) Pub. No.: US 2026/0224863 A1
(43) Pub. Date: Aug. 6, 2026

(54) BALLOON AND BALLOON CATHETER

(71) Applicant: TOKAI MEDICAL PRODUCTS, INC., Aichi (JP)

(72) Inventors: Katsumi TSUYUKI, Aichi (JP); Tatsumichi FUJIWARA, Aichi (JP)

(73) Assignee: TOKAI MEDICAL PRODUCTS, INC., Aichi (JP)

(21) Appl. No.: 19/152,475

(22) PCT Filed: Feb. 14, 2024
(86) PCT No.: PCT/JP2024/005110

§ 371 Date: Jul. 31, 2025

(30) Foreign Application Priority Data
Feb. 16, 2023 (JP) 2023-022477

(51) Int. Cl. A61M 25/10

(52) U.S. Cl. CPC A61M 25/104

(57) ABSTRACT

[Problem] The present invention provides a balloon for a catheter and a balloon catheter, whereby the risk of a blade becoming stuck in a lesion is reduced while the cutting performance is ensured. [Solution] A balloon 30 for use in a balloon catheter 100 according to the present invention is characterized in that, in the balloon 30 to be used in a balloon catheter, one or a plurality of cutting blades 40 are provided on the outer circumferential surface of the balloon 30 in parallel to the longitudinal direction of the balloon 30, and an end forming part 43c is formed at one end in the longitudinal direction of each of the cutting blades 40, the end forming parts 43c being formed so as to be higher than the height of the ends of the cutting blades 40 and to cover respective blade edges near the ends.

### TECHNICAL FIELD

The present invention relates to a balloon and a balloon catheter.

### BACKGROUND ART

In the treatment of a vascular lesion, for example, in percutaneous transluminal coronary angioplasty, a balloon catheter is used to expand a lesion of a coronary artery and improve blood flow. In addition, the balloon catheter may be used when performing percutaneous transluminal angioplasty (PTA) for treating arteriosclerosis occurring in the femoral artery, the iliac artery, the popliteal artery, the below-knee artery, or the like of the lower limb or vascular access interventional therapy (VAIVT) for treating stenosis or occlusion of upper limb vascular access.

However, when arteriosclerosis progresses, a plaque may be generated in the lesion, or the lesion may be further calcified to generate a hard portion. Such a lesion may not be able to expand the inside of the blood vessel only by the expansion force of the balloon catheter. As a device that enables treatment even in such a case, a cutting balloon catheter provided with a blade on a balloon is known. The cutting balloon catheter has a blade-like blade on the outer periphery of the balloon, and the blade can crack a calcified site to easily expand a lumen such as a lesion. Patent Literature 1 discloses the balloon catheter having such a blade, for example.

The balloon catheter according to Patent Literature 1 includes a balloon having a high elastic modulus and a high rigidity material connected to a catheter installed near a lesion in a blood vessel, and the balloon has a flexible cutting blade capable of changing the number, position, and the like. Due to the increase in flexibility, it is possible to improve catheter traceability and delivery property in a tortuous living body, and it is possible to advance the catheter on the guide wire to a lesion such as a cardiovascular cavity that is difficult to reach with a low flexibility catheter, and to excise the lesion.

However, in the cutting blade according to Patent Literature 1, since the metal blade is provided up to the end in the longitudinal direction, there is a possibility that the end of the metal blade strongly pierces and is stuck to a lesion such as plaque or calcification. In addition, there is a problem that the end of the metal blade may come into contact with a blood vessel other than the lesion at the time of inserting or removing the balloon and may be damaged.

### CITATION LIST

### Patent Literature

Patent Literature 1: JP 2008-519654 A

### SUMMARY OF INVENTION

### Technical Problem

Therefore, the present invention has been made to solve the above-described problems, and an object of the present invention is to provide a balloon for a catheter and a balloon catheter that reduce a possibility of a blade being stuck to a lesion while securing cutting performance.

### Solution to Problems

In order to achieve the object described above, the balloon and the balloon catheter according to the present invention employ the following means.

In a balloon for use in a balloon catheter according to the present invention, one or a plurality of cutting blades is provided on an outer peripheral face of the balloon in parallel with a longitudinal direction of the balloon, and wherein an end forming portion formed at a height same as a height of an end of the cutting blade or higher than the height of the end of the cutting blade is provided at either end of the cutting blade in the longitudinal direction.

By providing the end forming portions at either or both ends of the cutting blade in the longitudinal direction, it is possible to prevent the end of the blade tip of the cutting blade from strongly piercing and being stuck to a lesion such as plaque or calcification, and it is possible to reduce the possibility that the end of the cutting edge of the cutting blade pierces and is stuck to the lesion. In addition, it is possible to prevent the possibility that the end of the cutting blade comes into contact with a blood vessel other than the lesion at the time of inserting or removing the balloon and is damaged.

In the balloon according to the present invention, the end forming portion may be formed so as to cover part of the end of the blade tip of the cutting blade.

By covering the end of the cutting blade, it is possible to further reduce the possibility that the blade tip of the cutting blade first pierces a lesion such as a plaque or calcification. Furthermore, since the end of the cutting blade that is most likely to fall off is covered when passing through the curved portion of the blood vessel, a possibility that the end of the cutting blade falls off from the balloon or the base portion can be reduced, and as a result, a possibility that the entire cutting blade is peeled off or falls off can be reduced.

Furthermore, in the balloon according to the present invention, the end forming portion may be formed so that the height gradually decreases toward the distal end. With such a configuration, it is possible to reduce the end forming portion being caught in the blood vessel when the balloon in the blood vessel is advanced or retracted, and to smoothly move the balloon.

Furthermore, in the balloon according to the present invention, the cutting blade may be attached to a base made of resin, and the end forming portion may be molded integrally with the base portion. By integrally forming the end forming portion and the base portion, it is possible to reduce the possibility that the end forming portion itself falls off.

Furthermore, in the balloon according to the present invention, the cutting blade may be divided into a plurality of parts in the longitudinal direction. By dividing the cutting blade into a plurality of parts, the balloon can be easily curved, and it is possible to more easily follow the curved blood vessel.

Furthermore, in the balloon according to the present invention, the cutting blade may include the metal blade and the resin blade alternately disposed. When a cutting blade includes a metal blade and a resin blade alternately disposed, it is possible to obtain a balloon having high flexibility as a whole due to flexibility of the resin blade and excellent blood vessel followability while securing cutting performance by the metal blade.

Furthermore, the present invention provides a balloon catheter having a balloon as described above. Such a balloon catheter can provide a balloon catheter having the above-described effects.

### BRIEF DESCRIPTION OF DRAWINGS

FIG. 1 is a side view schematically illustrating one of embodiments of a balloon catheter 100 according to the present invention.

FIG. 2 is a side view schematically illustrating a balloon 30 of the balloon catheter 100 according to the present invention.

FIG. 3 is a side view schematically illustrating a cutting blade 40 used in the balloon catheter 100 according to the present invention.

FIG. 4 is a side view schematically illustrating another embodiment of the cutting blade 40 of the balloon catheter according to the present invention.

FIG. 5 is a side view schematically illustrating still another embodiment of the cutting blade 40 of the balloon catheter according to the present invention.

FIG. 6 is a schematic view illustrating a use state of the balloon catheter according to the present invention.

### DESCRIPTION OF EMBODIMENTS

Hereinafter, an embodiment of a balloon catheter 100 according to the present invention will be described with reference to the drawings.

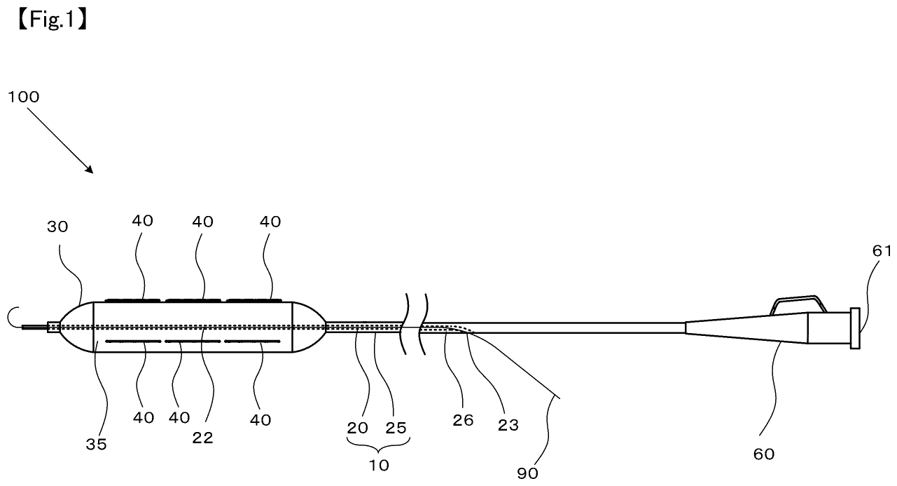

FIG. 1 is a side view schematically illustrating a balloon catheter 100 according to an embodiment. A balloon catheter 100 according to the present invention mainly includes a catheter shaft 10, the balloon 30, and an operation member 60 provided at a proximal end of the balloon catheter 100. In each figure, a cutting blade 40 is drawn larger than the actual scale with respect to the size of the balloon for ease of illustration. In addition, since the description of the balloon catheter 100 includes the description of the balloon 30 according to the present invention, the description of the balloon is replaced with the description of the balloon catheter 100. Note that, in the present embodiment, a description will be given using a rapid exchange (RX) type, but an over-the-wire type may be used.

As illustrated in FIG. 1, the catheter shaft 10 mainly includes an inner tube 20 and an outer tube 25 provided on the proximal side of the balloon 30. The inner tube 20 is inserted from the distal end of the balloon 30 to the proximal side and communicates with a guide wire port 23 manufactured on the side of the catheter shaft 10.

The inner tube 20 has a guide wire lumen 22 inside through which a guide wire 90 passes for carrying the balloon 30 of the balloon catheter 100 to a lesion of a patient or for injecting a medicinal solution or the like. Other configurations are not particularly limited. As the material used for the inner tube 20, for example, polyamide, polyvinyl chloride, polyethylene, polyurethane, a polyether block amide copolymer, polyethylene terephthalate, an olefin-based polymer such as polypropylene, or a copolymer formed of a combination thereof can be used, but the material is not limited thereto. Preferably, a thermoplastic resin that has been used in medical applications such as polyamide and polyurethane may be used.

The outer tube 25 is a tube provided on the proximal side with respect to the balloon 30 and on the outer peripheral side of the inner tube 20. As illustrated in FIG. 1, an inflation fluid lumen through which an inflation fluid lumen 26 for balloon inflation flows is formed between the inner tube 20 and the outer tube 25. The inflation fluid lumen 26 communicates with the balloon lumen 35 in the balloon 30, and the balloon 30 is inflated by the inflation fluid flowing through the inflation fluid lumen 26.

The balloon 30 is a hollow member provided at or near the distal end of the catheter shaft 10 and having a balloon lumen 35 inside, and can be inflated by flowing an inflation fluid inside. The balloon 30 may be a non-stretchable material or a material having low stretchability. As a material of the balloon 30, for example, nylon (registered trademark), polyvinyl chloride, polyethylene, polyurethane, a polyether block amide copolymer, polyethylene terephthalate an olefin-based polymer such as polypropylene, or a copolymer formed of a combination thereof can be used, but the material is not limited thereto. Preferably, a thermoplastic resin that has been used in medical applications such as polyamide and polyurethane may be used.

The balloon 30 and the outer tube 25 may be integrally molded, or may be separately molded and then joined. Further, the balloon 30 and the outer tube 25 may be made of the same material or different materials.

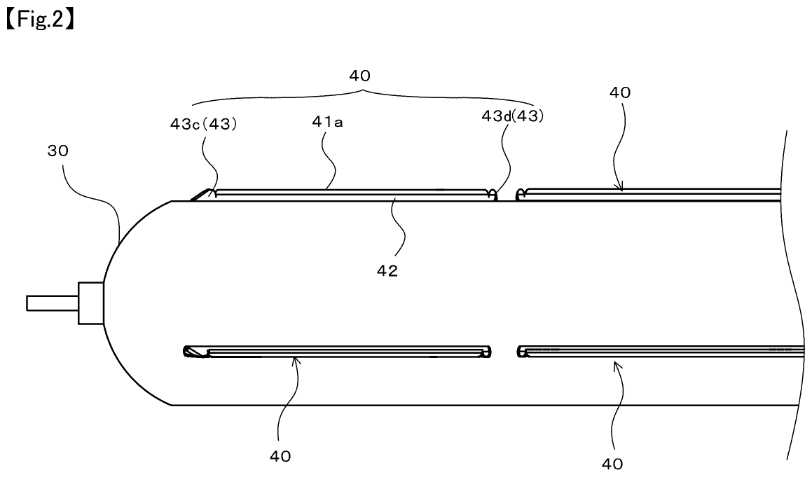

As illustrated in FIG. 2, one or a plurality of cutting blades 40 is attached to the outer peripheral face of the balloon 30. The cutting blade 40 is provided in parallel with the longitudinal direction of the balloon 30. The cutting blade 40 has a blade on the outer peripheral side, and for example, by inflating the balloon 30 at a plaque formed in a blood vessel due to arteriosclerosis or the like or a calcified site in the blood vessel, a crack can be made with the blade at the plaque or the calcified site, and the blood vessel lumen of the lesion and the like can be easily expanded.

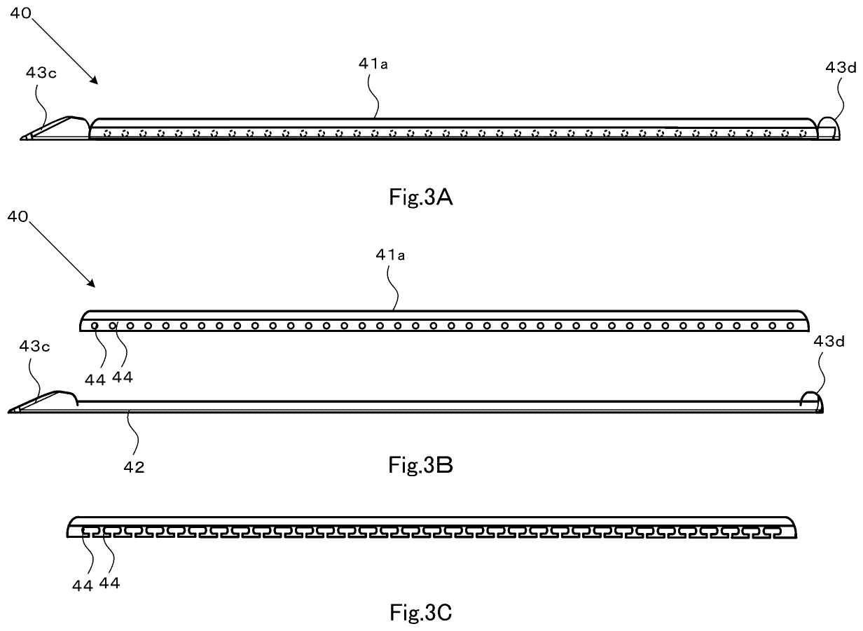

As illustrated in FIG. 3A, the cutting blade 40 is manufactured by integrally molding a metal blade 41a with a resin base portion 42 attached to the balloon 30. As illustrated in FIG. 3B, the metal blade 41a has a plurality of round holes 44 at a portion embedded in the base portion 42. Since the resin flows in the round hole 44 by providing the round hole 44, it is possible to prevent the metal blade 41a from falling off from the base portion 42. In addition, as illustrated in FIG. 3C, instead of the round hole 44, a cutout portion 44a formed so that the bottom face side is narrow and the depth side is wide may be used. By forming the cutout portion 44a, the metal blade 41a can be prevented from falling off from the base portion 42, and the metal blade 41a can be easily curved. The base portion 42 has end forming portions 43 at either one end or both ends. That is, the end forming portion 43 (43c, 43d) is provided at one end or both ends of the cutting blade 40 according to the present embodiment in the longitudinal direction. By disposing the end forming portions 43c and 43d at the end of the cutting blade 40, the blade of the cutting blade 40 is prevented from being disposed at the end, so that it is possible to prevent the possibility that the end of the cutting blade 40 first strongly pierces and is stuck to a lesion such as plaque or calcification. In addition, it is possible to prevent the end of the cutting blade 40 from coming into contact with a blood vessel other than a lesion and being damaged when the balloon 30 is inserted or removed. The end forming portion 43 may be formed so as to have a height same as a height of a blade of the cutting blade 40 or higher than the height of the blade of the cutting blade 40. By forming the end forming portion 43 to be equal to or higher than the height of the blade of the cutting blade 40, it is possible to further reduce the possibility that the end of the cutting blade 40 pierces a lesion such as plaque or calcification. In addition, it is possible to prevent the possibility that the end of the cutting blade 40 comes into contact with a blood vessel other than the lesion and is damaged when the balloon 30 is inserted or removed. In addition, the form of the end forming portions 43c and 43d is not particularly limited, and the distal end may be formed in a substantially hemispherical shape as in the end forming portion 43d. Preferably, as in the end forming portion 43c, it is preferable to form so that the height gradually decreases toward the tip side and is thinner. The end forming portion 43c whose height gradually decreases toward the distal end and is thinner may be provided at the most front end and the most rear end of the plurality of cutting blades 40 disposed in the longitudinal direction. By configuring the distal end to be thin in this manner, it is possible to smoothly move the end forming portion 43 without being caught in the blood vessel when the balloon in the blood vessel is advanced or retracted. The end forming portion 43 may be formed so as to cover part of the blade tip of the cutting blade 40. As a result, the possibility that the cutting blade 40 falls off can be reduced.

Attachment of the cutting blade 40 to the base portion 42 is not limited to the above-described integral molding, and can be performed by an appropriate method such as adhesion, casting, thermal bonding, mechanical coupling, welding, or brazing, or another appropriate method. Attachment of the base portion 42 to the balloon 30 can be similarly performed by an appropriate method such as bonding, casting, thermal bonding, mechanical coupling, welding, brazing, or the like, or another appropriate method.

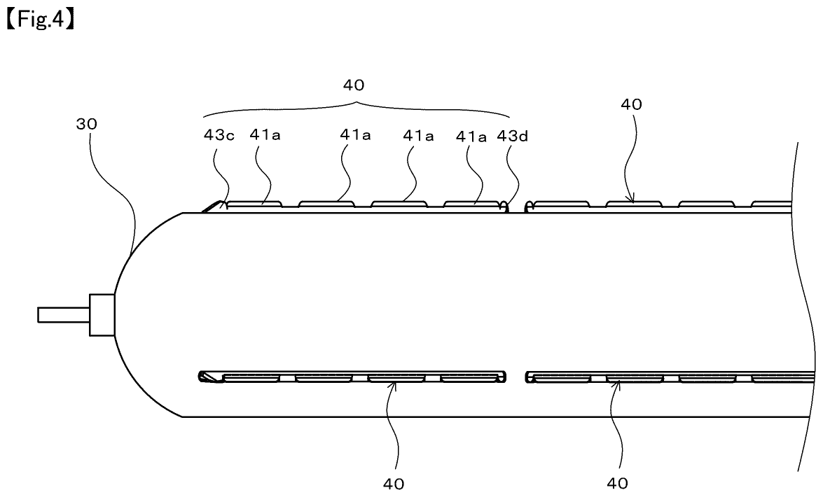

The number of the cutting blades 40 is not particularly limited, and may be one or more. As illustrated in FIG. 2, a plurality of the cutting blades 40 may be provided in the circumferential direction, or a plurality of the cutting blades may be provided in the longitudinal direction. Further, as illustrated in FIG. 4, each cutting blade 40 may be formed in a divided manner.

As described above, by forming the cutting blade 40 in a divided manner, the flexibility is improved and the blood vessel followability can be improved.

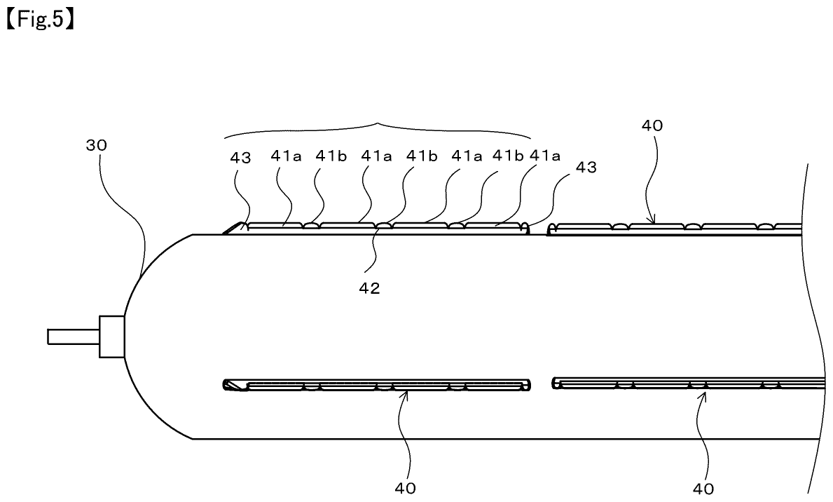

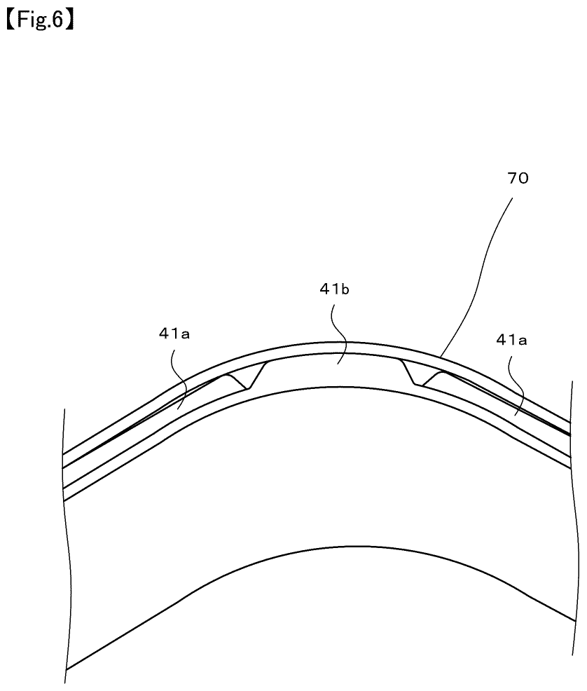

Further, as illustrated in FIG. 5, the metal blade 41a and the resin blade 41b may be alternately disposed. In general, the metal blade 41a is hard and excellent in cutting performance, but has low flexibility in curving or extending, and in a curved blood vessel or the like, it is difficult for the metal blade 41a to follow the inner face of the blood vessel or being advanced in the blood vessel. When trying to forcibly follow the balloon, the metal blade 41a may be broken or the metal blade 41a may be peeled off from the balloon 30. On the other hand, since the resin blade 41b is excellent in flexibility and has high flexibility, it is easy to follow the inner face of a curved blood vessel and has characteristics of being excellent in delivery property. Therefore, by alternately disposing the metal blade 41a and the resin blade 41b, it is possible to secure the followability of the blood vessel by the flexibility of the resin blade 41b while securing the cutting performance of the metal blade 41a as illustrated in FIG. 6. In addition, since the lengths of the metal blade 41a and the resin blade 41b are shortened by alternately disposing, it is easy to follow the curved blood vessel from this point. As a result, there is an effect that the balloon 30 can be advanced to a position that is difficult to reach with another cutting balloon catheter having low flexibility, and followability to the inner face of the blood vessel is improved.

The lengths of the metal blade 41a and the resin blade 41b are not particularly limited, but when the length of the metal blade 41a is longer than that of the resin blade 41b, the cutting performance is improved. On the other hand, when the length of the metal blade 41a is formed shorter than that of the resin blade 41b, the shorter the metal blade 41a is likely to follow the curve in the blood vessel, and the longer the resin blade 41b is likely to follow the curve in the blood vessel.

Preferably, the length of the metal blade 41a is 1.0 mm to 40 mm, and the length of the resin blade 41b is 0.5 mm to 18 mm.

The operation member 60 has an inflation port 61 into which a balloon inflation fluid is inserted, and the balloon can be inflated by inserting the balloon inflation fluid from the inflation port.

The balloon catheter 100 prepared as described above is used as follows. First, the guide wire 90 is inserted into the lesion. Thereafter, the balloon 30 is advanced on the guide wire 90 to pass through a blood vessel 70 to reach the lesion. Next, an inflation fluid is injected into the balloon lumen 35 through the inflation fluid lumen 26 to inflate the balloon 30. With this inflation, as illustrated in FIG. 6, the cutting blade 40 expands the blood vessel 70 while excising the lesion, and treats the blood vessel 70.

Note that the present invention is not limited to the above-described embodiments at all, and it goes without saying that the present invention can be implemented in various modes as long as they fall within the technical scope of the present invention.

### INDUSTRIAL APPLICABILITY

As shown in the embodiments described above, a balloon catheter for lumen expansion can be industrially provided.

### REFERENCE SIGNS LIST

10 . . . catheter shaft
20 . . . inner tube
22 . . . guide wire lumen
23 . . . guide wire port
25 . . . outer tube
26 . . . inflation fluid lumen
30 . . . balloon
35 . . . balloon lumen
40 . . . cutting blade
41a . . . metal blade
41b . . . resin blade
42 . . . base portion
43 . . . end forming portion
43c . . . end forming portion
43d . . . end forming portion
44 . . . cutout portion
60 . . . operation member
61 . . . inflation port
70 . . . blood vessel
100 . . . balloon catheter

1. A balloon for use in a balloon catheter, wherein one or a plurality of cutting blades is provided on an outer peripheral face of the balloon in parallel with a longitudinal direction of the balloon, and wherein an end forming portion formed at a height same as a height of an end of the cutting blade or higher than the height of the end of the cutting blade is provided at either end of the cutting blade in the longitudinal direction.

2. The balloon according to claim 1, wherein the end forming portion is formed so as to cover part of an end of a blade tip of the cutting blade.

3. The balloon according to claim 1, wherein the end forming portion has a height gradually decreasing toward a distal end.

4. The balloon according to claim 1, wherein the cutting blade is attached to a base portion made of resin, and the end forming portion is molded integrally with the base portion.

5. The balloon according to claim 1, wherein the cutting blade is divided into a plurality of parts in the longitudinal direction.

6. The balloon according to claim 1, wherein the cutting blade includes metal blades and resin blades that are alternately disposed.

7. A balloon catheter comprising the balloon according to claim 1.

8. A balloon catheter comprising the balloon according to claim 2.

9. A balloon catheter comprising the balloon according to claim 3.

10. A balloon catheter comprising the balloon according to claim 4.

11. A balloon catheter comprising the balloon according to claim 5.

12. A balloon catheter comprising the balloon according to claim 6.
